(19) United States
(12) Patent Application Publication
(10) Pub. No.: US 2023/0067681 A1
(43) Pub. Date: Mar. 2, 2023

(54) TRAINING SYSTEM AND METHODS FOR MIXING OF PHARMACEUTICAL COMPONENTS

(71) Applicant: JANSSEN PHARMACEUTICA NV, Beerse (BE)

(72) Inventor: Ian Scrimgeour, East Lothian (GB)

(21) Appl. No.: 17/895,399

(22) Filed: Aug. 25, 2022

Related U.S. Application Data
(60) Provisional application No. 63/238,106, filed on Aug. 27, 2021.

(51) Int. Cl. G09B 23/28; G09B 5/02

(52) U.S. Cl. CPC G09B 23/285; G09B 5/02

(57) ABSTRACT

A device and system for training users in a proper mixing of pharmaceutical components. The devices comprise a housing including a simulated plunger, finger flange, and barrel, a removeable clip for coupling to a syringe, and a smart label for affixing to a syringe. A microcontroller receives motion information from an accelerometer and orientation information from a magnetometer to assess motion, frequency, and orientation of shaking against predetermined parameters. A vibration meter that is spaced apart from the magnetometer provides haptic feedback to the user. A display unit displays information regarding the satisfaction of the shaking parameters to the user.

### CROSS REFERENCE TO RELATED APPLICATIONS

This application claims priority to U.S. Provisional Application No. 63/238,106 filed on Aug. 27, 2021, the entire contents of which are expressly incorporated herein by reference.

### BACKGROUND

Some pharmaceutical products are provided in two or more liquid and/or solid phases for agitation, such as manual shaking, before injection or like administration. For example, a pharmaceutical product may be provided as particles suspended in a liquid. Health Care Professionals (HCPs), and in some cases patients or caregivers, responsible for agitating the separate phases (for example to resuspend a suspension) may provide insufficient or inconsistent agitation because they are unfamiliar with the pharmaceutical product, have experience agitating other pharmaceutical products that have dissimilar shaking requirements, mistime the shake duration, and/or underestimate the vigor required to adequately mix the product.

U.S. Pat. Nos. 11,037,465 and 10,861,351 and U.S. Patent Publications US2018/0190153 and US2018/0190154, entitled, “Devices And Methods For Drug Administration And Mixing, And Training Of Proper Techniques Therefor,” disclose devices and methods for training users in desired mixing of pharmaceutical components, or for aiding or performing the mixing. The patents disclose a stand-alone, training device, referred to as a mimic trainer, that has a form factor close to that of the syringe so it may represent the experience of shaking the real syringe as closely as possible. The stand-alone device provides the user an opportunity to shake a device and learn what level of vigor is required when they come to shake the real device.

The mimic trainer may include an accelerometer and a microcontroller for determining whether the magnitude of the motion and orientation of the housing are sufficient with respect to predetermined thresholds, including magnitude of the force applied during the shaking, the orientation of the housing and duration of such shaking; and announcing via the user notification device as to whether the motion or orientation of the housing being shaken during one of a drug administration or a training event meets the predetermined thresholds. The patents disclose other embodiments, such as a housing for holding an administration device, such as a syringe, having the above capabilities.

### SUMMARY

A system for training a user, such as an HCP, in proper mixing of pharmaceutical components includes an injection training device that does not include the syringe, but rather serves as a stand-in for a syringe for training purposes. Alternatively, the system may clip onto an actual syringe or other injection device. The system includes the functionality of training the user and providing feedback to the user regarding the sufficiency of mixing of the pharmaceutical components. In the instance of a system that clips onto an actual syringe, the system can also be used to provide feedback to the user regarding the sufficiency of mixing of actual pharmaceutical components prior to administration. The system also includes a display device for providing information to a user during training. The system may be employed to simulate the proper mixing of two or more components of a pharmaceutical product supplied in a syringe.

The injection training device includes a housing, a controller disposed in the housing, an accelerometer in communication with the controller, and (optionally) a magnetometer in communication with the controller. The accelerometer is configured to detect and output, to the controller, motion information regarding motion of the housing. The magnetometer is configured to detect and output, to the controller, orientation information regarding orientation of the housing. Alternatively, the injection training device could include a smart label/flexible circuit adhered to a syringe.

Thus, according to a first aspect, A system for training a user in proper mixing of pharmaceutical components in a syringe, the system comprises: an injection training device including: a housing; a controller disposed in the housing; one or more motion sensors in communication with the controller, wherein the one or more motion sensors is configured to detect and output, to the controller, (i) motion information regarding motion of the housing and (ii) orientation information regarding orientation of the housing; and a display unit that is spaced apart from the injection training device, the display unit being in communication with the controller; wherein, when the housing is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

According to a second aspect, a system for training a user in proper mixing of pharmaceutical components in a syringe, the system comprises: an injection training device including: a flexible PCB affixed to a drug injection device; a controller disposed on the PCB; one or more motion sensors disposed on the PCB in communication with the controller, wherein the one or more motion sensors is configured to detect and output, to the controller, (i) motion information regarding motion of the syringe and (ii) orientation information regarding orientation of the syringe; and a display unit that is spaced apart from the injection training device, the display unit being in communication with the controller; wherein, when the syringe is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

According to a third aspect, a system for training a user in proper mixing of pharmaceutical components in a syringe, the system comprises: a clip assembly including: a housing adapted to be removably affixed to a drug injection device; a controller disposed in the housing; one or more motion sensors in communication with the controller, wherein the one or more motion sensors is configured to detect and output, to the controller, (i) motion information regarding motion of the housing and (ii) orientation information regarding orientation of the housing; and a display unit that is spaced apart from the injection training device, the display unit being in communication with the controller; wherein, when the housing is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

The corresponding method includes the steps of shaking an injection training device that includes a controller and one or more sensors; receiving, into the controller, motion data points from the one or more sensors in response to the shaking step; receiving, into the controller, orientation data points from the one or more sensors in response to the step of shaking the injection training device; classifying each one of the motion data points as satisfactory or unsatisfactory based on whether the motion data point meets a predetermined motion threshold; classifying each one of the orientation data points as satisfactory or unsatisfactory based on whether the orientation data point meets a predetermined orientation threshold, determining whether at least one characteristic of the shaking is satisfactory based on (i) a predetermined quantity of satisfactory motion data points meeting a predetermined threshold and/or (ii) a predetermined quantity of satisfactory orientation data points meeting a predetermined threshold; and based on the step of determining whether at least one characteristic of the shaking is satisfactory, displaying, on a display unit that is spaced apart from the injection training device, a visual indication of whether the at least one characteristic is satisfactory, and providing coaching tips to the user in real time.

### BRIEF DESCRIPTION OF THE FIGURES

FIG. 1 is a top, front perspective view of an injection training device assembly;

FIG. 2 is atop, rear perspective view of the injection training device of FIG. 1, showing a data transmission port;

FIG. 3 is a partially exploded view of the injection training device assembly of FIG. 1;

FIG. 4 is a top perspective view of the injection training device assembly of FIG. 1, with a portion of the housing removed to illustrate a printed circuit board and vibration motor assembly;

FIG. 5 is a bottom, front perspective view of the injection training device assembly of FIG. 3;

FIG. 6 is a rear elevation view of the injection training device assembly of FIG. 3;

FIG. 7 is a side elevation view of the injection training device assembly of FIG. 3;

FIG. 8 is a front elevation view of the injection training device assembly of FIG. 3;

FIG. 9 is an enlarged exploded view of a portion of the injection training device assembly shown in FIG. 8, with an upper portion of the housing removed;

FIG. 10 is a schematic of the injection device training system, including the injection training device assembly of FIGS. 1 and 2 and a display unit;

FIG. 11 is a schematic image illustrating the user shaking the injection training device of FIG. 1;

FIG. 12 is a schematic view of the display unit indicating that a parameter of the shaking by the user, illustrated in FIG. 11, is within a desired range;

FIG. 13 is a schematic view of the display unit alerting the user that a parameter of the shaking, illustrated in FIG. 11, is insufficient;

FIG. 14 is a schematic view of the display unit alerting the user that another parameter of the shaking, illustrated in FIG. 11, is insufficient;

FIG. 15 is a schematic view of the display unit indicating that the shaking of the injection training device, illustrated in FIG. 11, is within an acceptable range for all parameters;

FIG. 16 is a perspective view of another embodiment of the present disclosure, wherein the injection training device assembly includes a clip assembly that can be affixed to a syringe or other injection device;

FIG. 17 is a partially schematic view of the embodiment of FIG. 16 with a portion of the housing shown as transparent;

FIG. 18 is an enlarged view of the structure shown in FIG. 17;

FIG. 19 is an opposing view of the structure shown in FIG. 18;

FIG. 20 is a schematic perspective view of a syringe having a flexible circuit affixed thereto according to a another embodiment of the present disclosure; and

FIG. 21 is an enlarged view of the flexible circuit isolated from the syringe of FIG. 21.

### DETAILED DESCRIPTION OF ILLUSTRATIVE EMBODIMENTS

According to an aspect of the present disclosure, an injection training system 10 includes an injection training device assembly 110 and a display unit 210. The injection training system 10 is for training a user, such as an HCP, in a proper mixing of pharmaceutical products, such as one or more components thereof, including liquids, soluble and/or insoluble particles, or a combination thereof.

Injection training device assembly 110 may include a housing assembly 120; a battery assembly 140; a controller, such as a microcontroller 150; one or more sensors; a vibration motor assembly 170; a lighting assembly 180, and a communications module 195. The sensor may consist of an accelerometer 156 alone. The one or more sensors may, alternatively, be an accelerometer 156 and a magnetometer 160, as explained more fully below. Thus, the term “one or more sensors” is used herein to refer either to an accelerometer (such as the type that is capable of outputting motion data and orientation data), both and accelerometer and a magnetometer (such that together they are capable of outputting motion data and orientation data), and like components.

It is understood that each electronic components of injection training device 110, such as microcontroller 150, accelerometer 156, magnetometer 160, and/or communications module 195 may be formed as its own chip, the functions associated with one or more components may be embedded in multiple-purpose chips, or any combination thereof. For example, a single chip may include the accelerometer and controller functions, or a single chip may perform the controller and communication package functions, and/or other combinations.

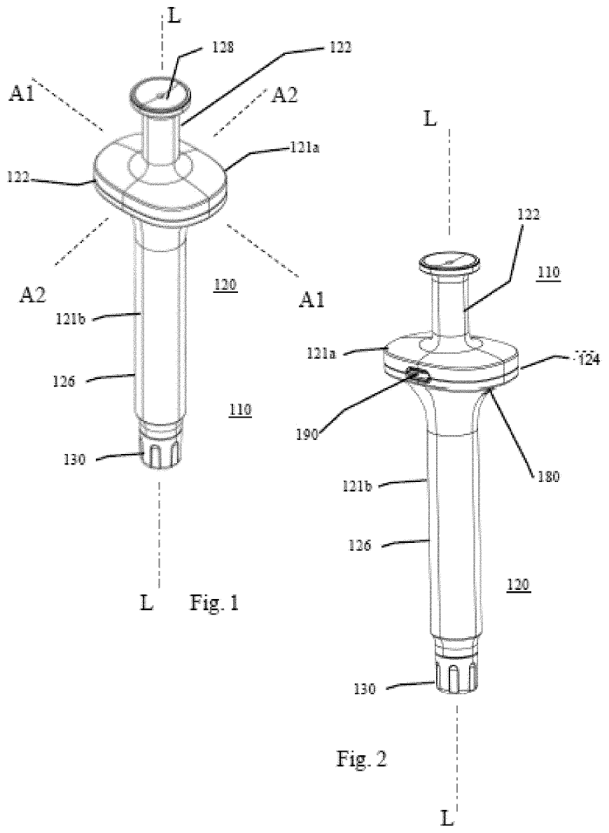

Housing 120 includes a plunger 122, a finger flange 124, and a barrel 126, each of which is configured along a longitudinal axis L-L, which is illustrated in FIGS. 1 and 2.

A recess 128 is formed in a proximal end portion of plunger 122 for providing a land for a user's thumb. The term “plunger” is used herein to refer to a rigid structure shown in the figures. Plunger 122 in the embodiment of the figures is not moveable relative to finger flange 124 and thus does not perform the function of a syringe plunger. Rather, plunger 122 generally represents or mimics the shape and location of a plunger of an injection device, however, other embodiments with a movable plunger 122 are possible.

Housing 120, in the embodiment shown in the figures, includes a plug or cap 130 at a distal tip of barrel 126 opposite housing plunger 122. Housing 120 may have a general shape and appearance of an injection device, such as a syringe, in which pharmaceutical products are provided. In this regard, injection training assembly may resemble or mimic the injection device in which the pharmaceutical components are provided.

Housing 120 extends longitudinally along an axis L-L, illustrated in FIGS. 1 and 2. Flange 124 is symmetrical about a plane defined by longitudinal axis L-L and major axis A1, and is symmetrical about another plane defined by longitudinal axis L-L and minor axis A2, as illustrated in FIG. 1. In the up-right orientation of FIGS. 1 and 2, each plane defined by the plane A1-A1—L-L and the plane A2-A2—L-L is vertical. The term “vertical,” as used herein refers broadly to the direction normal to the horizon, such as within the range +/−2 degrees of a vertical line, +/−10 degrees of a vertical line, +/−20 of a vertical line, or +/−30 degrees of a vertical line.

Barrel 126 extends downwardly from an underside of flange 124. Barrel 126 may be a right-angle cylinder that has a centerline that is coincident with longitudinal axis L-L. Barrel 126 may have other shapes, such as a non-circular cross-section.

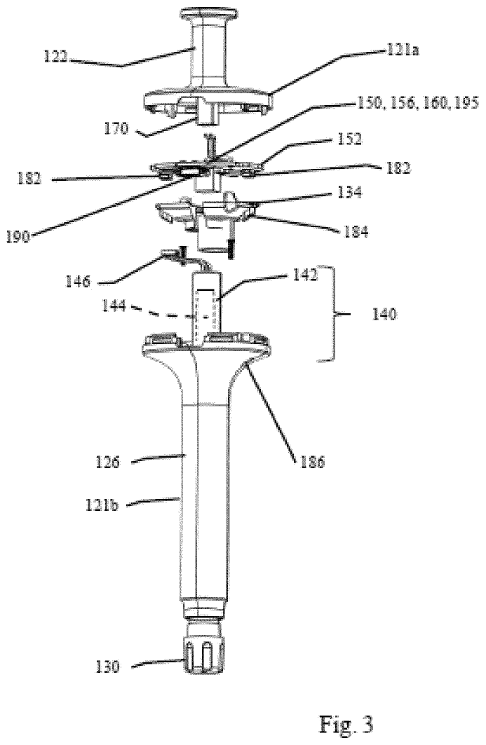

Housing 120 may be formed of any materials and methods, such as by an injection molded thermoplastic. FIG. 3 illustrates that housing 120 may be formed of molded components, including a housing upper component 121a, a housing lower component 121b, and a base structure 134. Housing lower component 121b may include at least the underside of housing flange 124, which may be formed integral with barrel 126. Upper component 121a may include a top side of flange 124 and plunger 122. Each one of housing upper and lower components 121a and 121b may include clips that may retain the components 121a and 121b together. Base structure 134 in the embodiment in the figures is enclosed within housing upper and lower components 121a and 121b. A lower portion of base structure 134 is cylindrical to receive battery assembly 140. An upper portion of base structure 134 supports printed circuit board 152, as explained more fully below.

Barrel 126 may be hollow so as to receive and contain battery assembly 140. As illustrated in FIG. 3, battery assembly 140 may include a battery holder or sleeve 142 to house one or more batteries 144, such as cylindrical batteries that may be disposable or rechargeable. Batteries 144 are illustrated in dashed lines to indicate that batteries 144 are housed within sleeve 142. Battery assembly 140 also may include a wiring unit 146 including wires and a connector that supply electrical power to PCB 152. Barrel 126 may include a longitudinal split and cylindrical fittings to engage plug 130 and base 134. Cap 130 may be removed to access batteries 144.

As explained above, the configuration of the embodiment of device 110 illustrated in FIGS. 1 and 2 may mimic the structure of an operational injection device for training purposes. In this regard, device 110 (in the exemplary embodiment shown of FIGS. 1 and 2) is configured such that (for example) a user may grasp housing 120 between the user's thumb on recess 128 and fingers on the underside of flange 124 with the user's index finger and middle finger straddling barrel 126. In this regard, housing 120 may be configured to mimic the grasping of an operational injection device, such as the type that requires shaking by a HCP or others for the preparation of a pharmaceutical product, such as, for example, paliperidone palmitate in formulations for 1 month, 3 month, or 6 month. Other hand positions and housing configurations are contemplated. For example, during a shaking motion of device 110, the user may grasp the barrel 126 between her thumb, index finger, and pointer finger with the cap pointing upward, e.g., generally in the direction of her head.

The controller may include or be a microcontroller 150 that may include a processor, memory, and programmable input and output means. The microcontroller 150 may be mounted on a printed circuit board (PCB) 152. Means to receive and control electrical power from battery assembly 140 may be mounted on PCB 152. Alternatively, the controller may be one or more separate microcontrollers, memory units, and input/output devices, and/or other conventional semiconductor components mounted on a PCB. Further, controller 150 may include the functionality of other components, such as accelerometer 156 and/or communication module 195.

A base 134 may be affixed within housing 120 via screws that pass through PCB 152 and into housing portion 121b, as illustrated in the figures. In this regard, base 134 is sandwiched between PCB 152 and housing lower portion 121b. In the embodiment in the figures, base 134 includes a grooved cylinder for being installed over battery sleeve 142.

Accelerometer 156 may comprise a conventional digital accelerometer, or alternatively may be an analog accelerometer having appropriate input and output means for communicating with microcontroller 150. In the embodiment of the figures, accelerometer 156 is mounted on PCB 152 and in communication with controller 150. Accelerometer 156 may be configured to detect the magnitude of acceleration forces applied to training device 110 along or about the longitudinal axis, as accelerometer may be a single axis, two axis, or three axis type. In this regard, an axis of accelerometer 156 may be aligned with the longitudinal axis L-L of the housing 120 such that measurements by that axis correspond to motion along the longitudinal axis of the housing 120. Alternatively, the accelerometer 156 may have at least two axes that are not aligned with the longitudinal axis of the housing 120 and the forces may be resolved in order to determine the motion along the longitudinal axis. The accelerometer 156 may also detect motion in any other direction, such as side-to-side motion, or rotation about any other axis. Further, accelerometer may be configured to sense the orientation of housing 120 relative to the earth's magnetic field before and during shaking and thus indicate whether longitudinal axis L-L of housing 120 is vertical, as well as the magnitude of the deviation of axis L-L from vertical, and whether the device 110 is oriented “tip up” or “tip down”.

In embodiments having a magnetometer in addition to the accelerometer, magnetometer 160 is in electrical communication with microcontroller 150 and mounted within housing 120, preferably on PCB 152. Magnetometer 160 may sense the orientation of housing 120 relative to the earth's magnetic field before and during shaking and thus indicate whether longitudinal axis L-L of housing 120 is vertical, as well as the magnitude of the deviation of axis L-L from vertical, and whether the device 110 is oriented “tip up” or “tip down”.

Vibration motor assembly 170 preferably includes an eccentric mass 172 and a DC motor 174 having a speed that is controlled by microcontroller 150 and/or display unit 210. Eccentric mass 172 is fixed on an output shaft of the DC motor 174. Thus, vibration motor assembly 170 in the embodiment of the figures is an eccentric rotating mass vibration motor (ERM). The speed of DC motor 174 is typically in the range of 6,000 to 12,000 rpm. The motor 174 may be pulsed on and off to provide a tactical guidance at a desired frequency, which frequency may be chosen to provide feedback to a user regarding the frequency at which device 110 is intended to be shaken. For example, if particular pharmaceutical components are ideally shaken at a frequency of 4 Hertz, a motor 174 operating at a speed of 8,000 rpm may be switched on for 100 ms, then off for 150 ms, which results in the 4 Hz pulse being felt by the user holding the housing 120. In this regard, there are four periods of operation of motor 174 (of 100 ms each) every second. The user thus may receive physical or haptic feedback of the desired shaking frequency and can shake faster or slower to match the desired frequency.

Vibration assembly DC motor 174 may be oriented such that its axis of rotation is coincident with housing longitudinal axis L-L, and located such that at least a part of the motor 174 is located within the plunger 122 of the housing 120. In the orientation of the figures, eccentric mass 172 is located in the upper portion (such as the upper half of a straight portion of the housing upper 121a) of plunger 122, which puts the eccentric mass close to a user's fingers when gripping device 110 and enables motor assembly 170 to be spaced apart from PCB 152 and magnetometer 160 to diminish electromagnetic interference, such as from motor 174 to magnetometer 160 and/or other electronic components. Also, electromagnetic shielding between the motor and magnetometer 160 and/or PCB may be employed.

A lighting assembly 180 may include a pair of light-emitting diodes (LEDs) 182 on opposing sides of housing 120. In the embodiment of the Figures, LEDs 182 are located on primary axis A1-A1. LEDs 182 may be mounted onto PCB 152. In the embodiment of the Figures, LEDs 182 are on the underside of PCB 152. Each LED 182 is aligned with a corresponding light guide 184 that extends through housing portion 121b to transmit light to a location that can be seen by a user.

Housing 120 also includes an opening through which a connection port 190, such as a conventional USB port of any type, an Apple lighting connector, or the like, may be accessed. Port 190 may be connected to controller 150 and/or battery assembly 140, such as for recharging batteries 144 (in embodiments in which batteries 144 are rechargeable), updating software, communicating with display device 210, and/or other functions as will be understood by persons familiar with communication technology.

Device 110 also may include a wireless communication module 195, such as the type that sends and receives data through standard communications protocols, such as a wireless personal area network, with display unit 210. The communication between display 210 and device 110 may be via a wireless communication protocol, such as Bluetooth Low Energy (“BLE”), Classic Bluetooth, Near Field Communication (“NFC”) or other wireless communication standard or customized communication means. Any of the communications protocol functions may be performed on either injection training device 110 and/or display unit 210. For example, display unit 210 may be configured to send a signal to microcontroller 150 upon establishing a communication link such that lightning system 180 lights to indicate that the device 110 and display 210 are in communication. Any handshaking, confirmation of the identity of device 110, security protocols, and/or like processes may be accomplished to initiate system 10. Moreover, any of the functions or process steps of system 10 may be embedded in software/application(s) running on device 110, display unit 210, and/or a combination thereof.

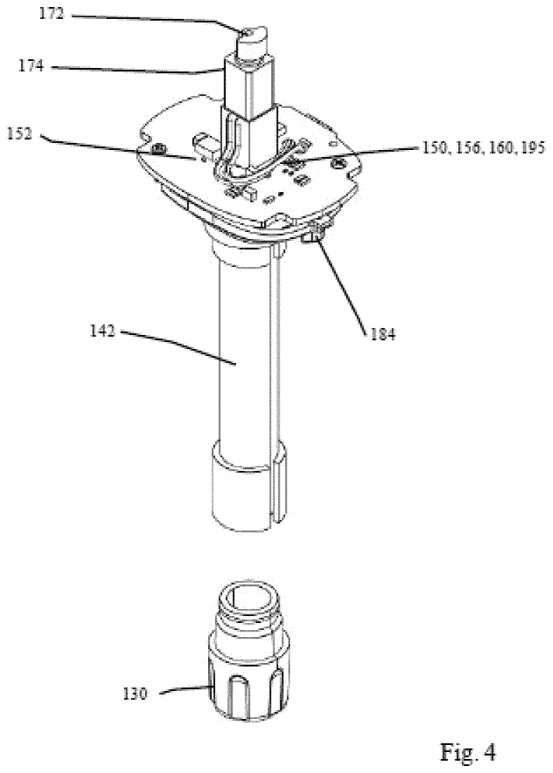

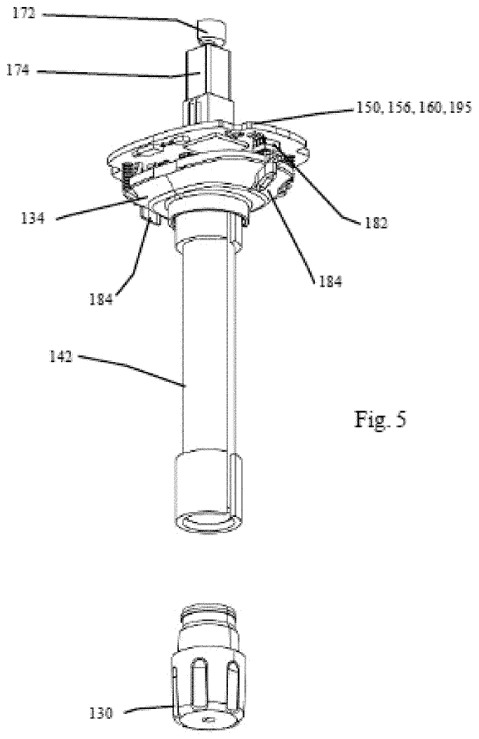

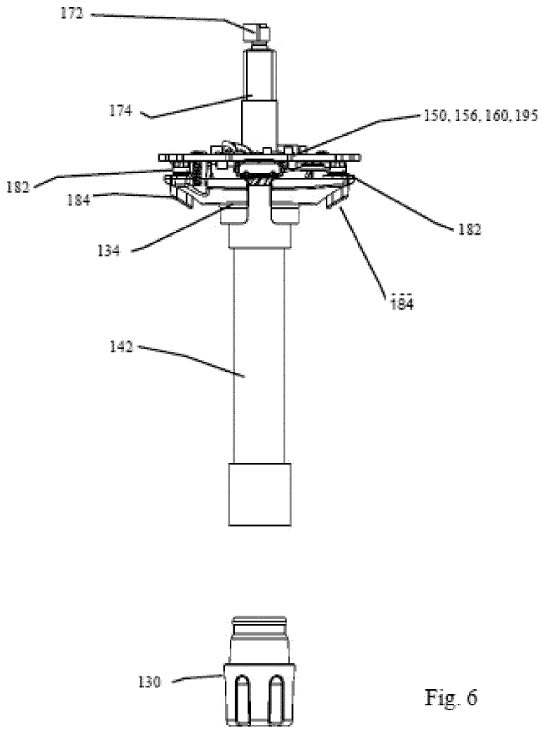

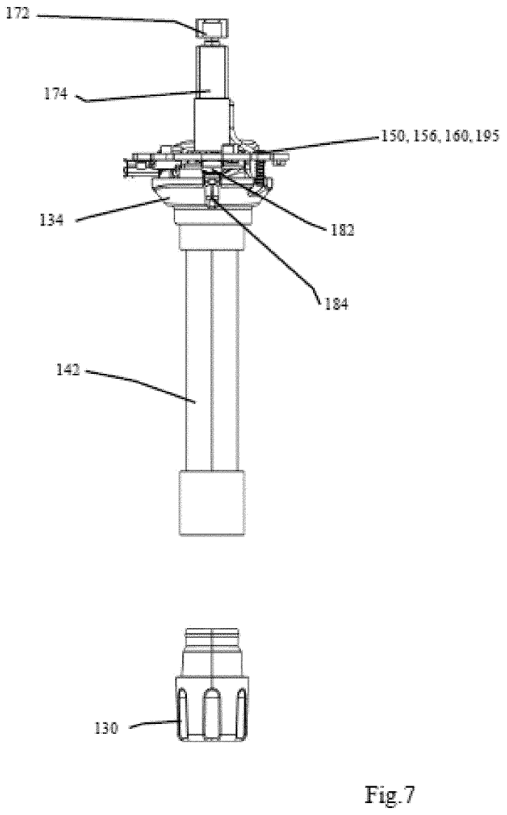

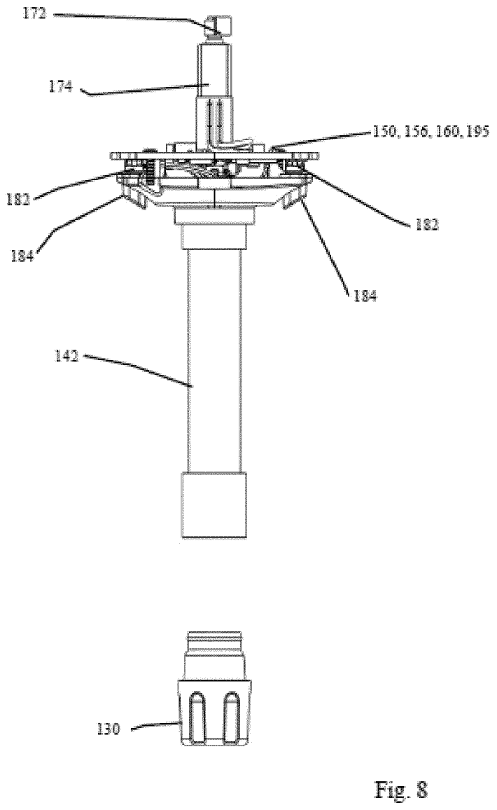

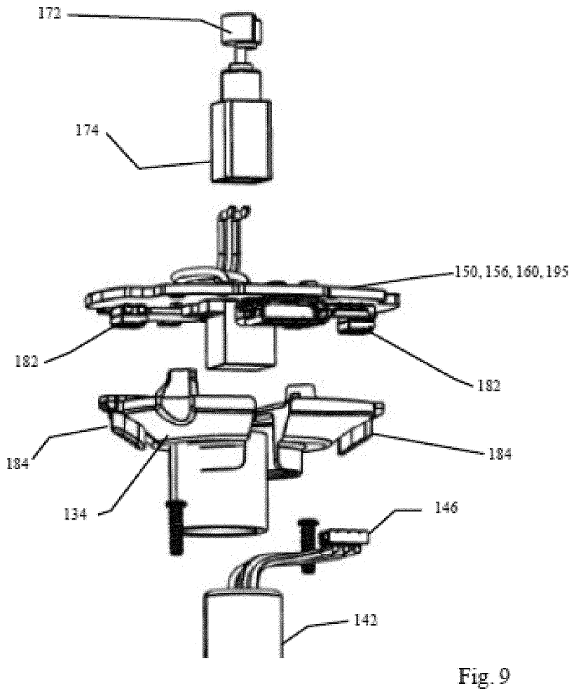

Wireless communication module 195 (or components thereof) may be mounted on PCB 152. FIGS. 3 through 9 use a single line for reference numbers 150, 156, 160, and 195 to indicate the location of the controller, accelerometer, magnetometer, and communications module, respectively. It is understood that the components may be anywhere within housing 120, such as laid out in an appropriate scheme on PCB 152 consistent with well-known electronic component design principles. A description of the operation of injection training device 110 and display unit 210 is provided below.

According to another aspect of the present disclosure, the training device assembly comprises a housing having a form factor that clips onto an actual drug administration device 900, for example, a syringe. According to an embodiment of the training system 300 shown in FIG. 16, housing 320 can clip onto the barrel and finger flange of drug administration device 900, which is a conventional pre-filled syringe, as explained more fully below. According to the embodiment of FIG. 16, the housing 320 can be removed from drug administration device 900 and reattached to another drug administration device 900, permitting the training system 300 to be re-used. In this embodiment, in addition to training a user on the proper mixing of pharmaceutical components, the training system can be used to provide feedback to a user as to whether actual pharmaceutical components in the drug administration device are sufficiently mixed prior to an actual administration (e.g., injection) of the drug.

In addition to the housing 320, the system 300 can include a display unit 210, similar to the embodiments described above. Administration device 900 may be a conventional syringe having a pharmaceutical product, such as one or more components thereof, including liquids, soluble and/or insoluble particles, or a combination thereof. The clip system 300 may be for training a user, such as a HCP, in a proper mixing of pharmaceutical products, such as one or more components thereof, including liquids, soluble and/or insoluble particles, or a combination thereof. As mentioned previously, system 300 may be used to notify a user that the contents of the drug administration device 900 are properly mixed and ready for administration.

Syringe 900 includes a plunger 922, a finger flange 924, and a barrel 926 that is adapted to contain components of a pharmaceutical product, such as one or more components thereof, including liquids, soluble and/or insoluble particles, or a combination thereof. Plunger 922 includes a shaft 930 and a thumb rest 932. Syringe 900 can comprise a conventional syringe known in the art.

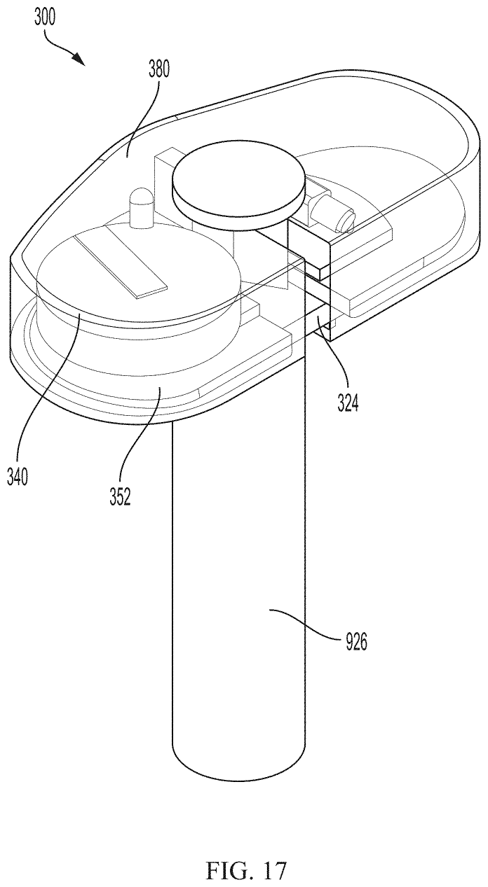

Clip assembly 310 includes a housing 320 that is adapted for interfacing with syringe 900 such that housing 320 is securely attached to syringe 900 and thus enable the functionality of some or all of injection training device 110, described above. In this regard, as illustrated in FIGS. 16 through 19, housing 320 has approximate C-shape that forms a recess 322 that extends longitudinally, as illustrated by longitudinal axis L-L in FIG. 16, and opens transversely (that is perpendicular to axis L-L). A slot 324 is formed in housing 320 such that slot 324 is transverse to axis L-L and accessible via recess 322.

Slot 324 is configured to receive finger flange 924 such that syringe 900 and clip assembly 310 are clipped together for the purpose of shaking to mix the pharmaceutical components. In this regard, shaft 930 of plunger 922 extends upwardly from housing 320 and barrel 926 extends downwardly from housing 320.

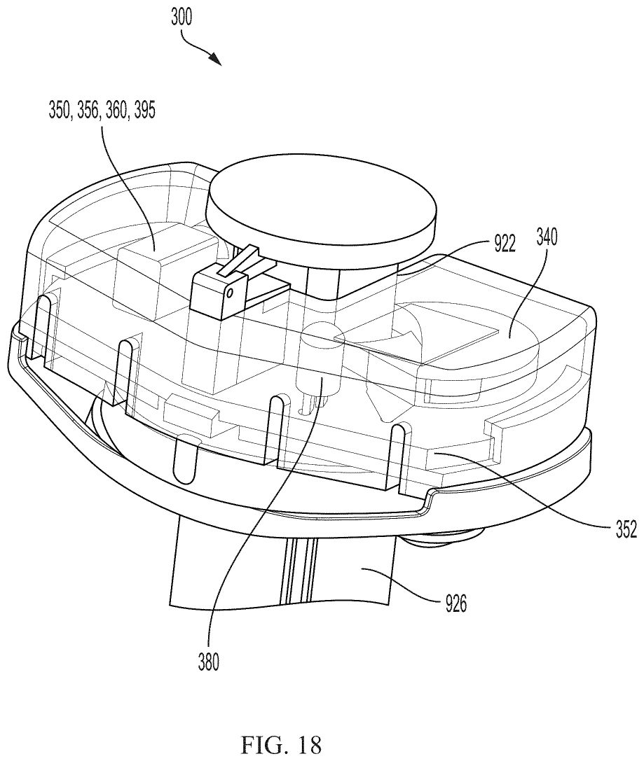

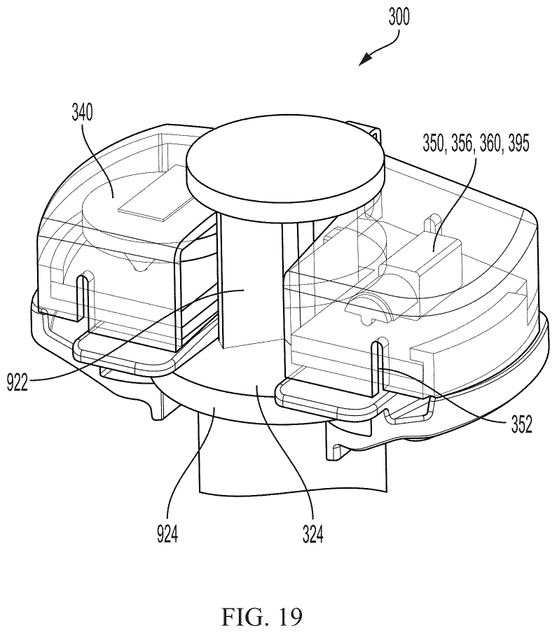

Clip assembly 310 may also include a battery 340, a PCB 352, a microcontroller 350, an accelerometer 356, (optionally) a magnetometer 360, and communications module 395, and a lighting system. In general, the components referred to by three numbers beginning with a 3 may have the same or similar structure and function as the corresponding elements referred to by three digits beginning with a 1. Thus, the functionality of electronic components 340, 352, 350, 356, 360, and 395 may be as described above for the corresponding elements of injection training device 110. It is understood that each electronic component of injection training device 310 may be formed as its own chip, the functions associated with one or more components may embedded as in multiple-purpose chips, or any combination thereof. In this regard, electronics components 350, 356, 360, and 395 are identified schematically in FIGS. 18 and 19 as a single package.

Microcontroller 350 that may include a processor, memory, and programmable input and output means. The microcontroller 350 may be mounted on a printed circuit board (PCB) 352 that is secured within housing 320. Means to receive and control electrical power from battery assembly 340 may be mounted on PCB 352 or otherwise connected. Alternatively, the controller may be one or more separate microcontrollers, memory units, and input/output devices, and/or other conventional semiconductor components mounted on a PCB. Further, controller 350 may include the functionality of other components, such as an accelerometer and/or communication module.

The accelerometer of 356 may have the structure, functionality, and connections with other components as described above in the description of accelerometer 156 of in injection device 110. Regarding its function, the accelerometer may be configured such that an axis of the accelerometer may be aligned with the longitudinal axis L-L of the syringe 900 such that measurements by that axis correspond to motion along the longitudinal axis of the housing syringe 900. Alternatively, the accelerometer may have at least two axes that are not aligned with the longitudinal axis L-L of syringe 900 and the forces may be resolved in order to determine the motion along the longitudinal axis. The accelerometer may also detect motion in any other direction, such as side-to-side motion, or rotation about any other axis. Further, the accelerometer of system 310 may be configured to sense the orientation of syringe 900 relative to the earth's magnetic field before and during shaking and thus indicate whether longitudinal axis L-L of syringe 900 is vertical, as well as the magnitude of the deviation of axis L-L from vertical, and whether the syringe 900 is oriented “tip up” or “tip down”.

In embodiments having a magnetometer in addition to the accelerometer, the magnetometer is in electrical communication with microcontroller 350 and may be mounted within housing 320, preferably on PCB 352. The magnetometer may sense the orientation of syringe 900 relative to the earth's magnetic field before and during shaking and thus indicate whether longitudinal axis L-L of syringe 900 is vertical, as well as the magnitude of the deviation of axis L-L from vertical, and whether the syringe 900 is oriented “tip up” or “tip down”.

Device 310 may include a vibration motor assembly (not shown in FIGS. 16 through 19) having the structure, function, and orientation of the vibration motor assembly 170 described for injection device 110. The vibration motor assembly of device 310 may be located at any convenient location within housing 320. The speed of the DC motor of the vibration motor assembly is typically in the range of 6,000 to 12,000 rpm. The motor may be pulsed on and off to provide a tactical guidance at a desired frequency, which frequency may be chosen to provide feedback to a user regarding the frequency at which syringe 900 is intended to be shaken, as described above for vibration motor assembly 170. In embodiments of clip assembly 310 having both a magnetometer and a vibration motor assembly, the magnetometer and vibration motor assembly may be spaced apart, such as on opposing (transverse) sides within housing 320 about plunger shaft 930.

The lighting assembly may include one or more light-emitting diodes (LEDs) 380 that may be mounted onto PCB 352. An aperture or lens 382 (shown in FIG. 16) in housing 320 exposes the light from LED 380 to the exterior of housing 320. A light guide or the like may be provided to enhance the transmission of light from LED 380 to outside housing 320 such that the light may be seen by the user. LED 380 may perform the same function as LED 180 described for injection training device 110.

A communication module 395 (schematically called out together with other electronic components in FIGS. 16-19) having the structure, function, and connections as described above for communication module 195 of injection device 110 may be provided. For example, a wireless communication module 395 may be of the type that sends and receives data, and communicates with display unit 210, using the protocols and technology as described above for communication module 195 for injection training device 110. Any of the communications protocol functions may be performed on either syringe clip assembly 310 and/or display unit 210. For example, display unit 210 may be configured to send a signal to microcontroller 350 upon establishing a communication link such that lighting system 380 lights to indicate that the clip assembly 310 and display 210 are in communication. Any handshaking, confirmation of the identity of clip assembly 310, security protocols, and/or like processes may be accomplished to initiate system 300. Moreover, any of the functions or process steps of system 300 may be embedded in software/application(s) running on clip assembly 310 (such as but not limited to electronics components thereof), display unit 210, and/or a combination thereof. A description of the operation of injection training device 110 and display unit 210 is provided below.

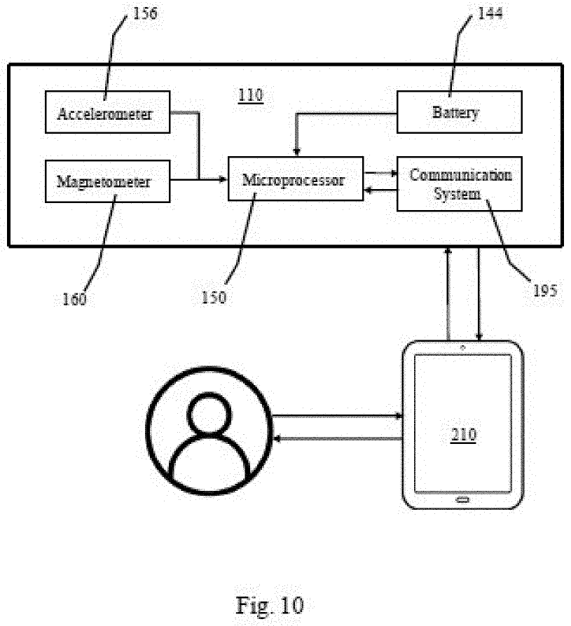

Patent Publication WO2021/095003, titled “Drug Delivery Device Sensing Modules,” which is incorporated herein in its entirety, discloses embodiments of drug delivery devices and other apparatus that incorporate thin-film sensing modules, for example, in the form of a label that can be adhered (e.g., with adhesive) or otherwise affixed to an existing drug delivery device (e.g., syringe). In other words, in place of the housing 120 and/or housing 320 shown and described in connection with FIGS. 1-10 and FIGS. 16-19, respectively, the electronic elements and their functions can be implemented into a thin-film label that can be adhered or otherwise affixed to a drug administration device, such as a syringe. The thin-film label may communicate with display unit 210 in the same or similar manner as described in previous embodiments, to provide user interaction, as described fully in the description of operation.

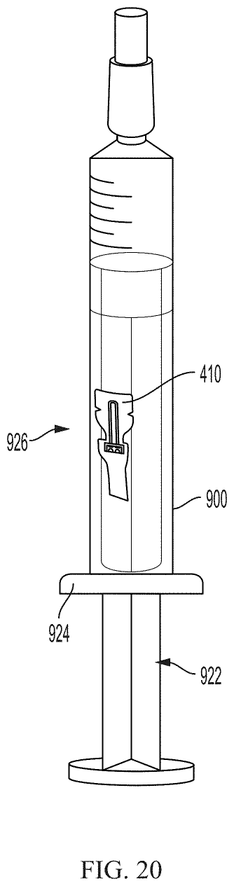

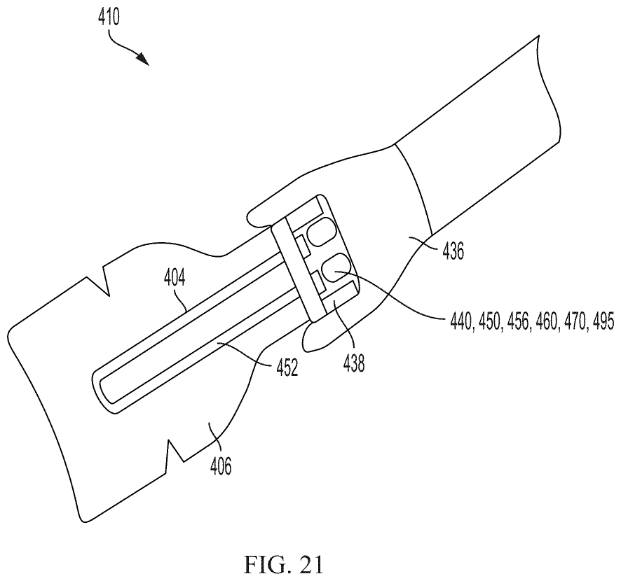

FIG. 20 illustrates a syringe 900 and thin-film label or smart-label 410. Label 410 can include a microcontroller 450, an accelerometer 456, and a communications module 495, as well as optional components magnetometer 460 and vibration motor 470, each of which is operationally coupled to a flexible PCB 452, as identified schematically in FIG. 21. Smart-label 410 may also include a battery for powering the electronic components.

As schematically illustrated in FIG. 20, label 410 is affixed to barrel 926 of syringe 900. Syringe 900 also includes a plunger 922 and a finger flange 924. The functionality of electronic components 440, 452, 450, 456, 460, and 495 may be as described above for the corresponding elements of injection training device 110. Label 410 may be aligned with the longitudinal axis of syringe 900.

In the context of the description above, injection training vice 110, clip assembly 310, and/or the syringe having smart label 410 may communicate with display unit 210 to detect whether the shaking motion and the orientation of the elements 120, 320, and 410 during shaking are sufficient relative to the desired parameters related to the shaking. In this regard, the convention 110/310/410 is employed where the description applies to any one of the devices or components of the corresponding, respective training system 10, clip assembly 310, and/or film apparatus 410.

The parameters may include one or more (or all) of the following, which are referred to as motion data: the magnitude of the force applied to the device 110/310/410 along the longitudinal axis L-L, acceleration of the device 110/310/410 throughout the shaking cycle, rate of shaking (such as reflected in shaking cycles per unit time), the magnitude of side-to-side shaking (that is, shaking not aligned with longitudinal axis L-L or normal to axis L-L), and/or the longitudinal shaking relative to the side-to-side shaking. The parameters may also include one or more of orientation of the housing 120, housing 320, and or film apparatus 410 and whether the device is oriented with the tip pointed upwardly—that is, “tip up,” which are referred to herein as orientation data. Total duration of the shaking, such as 60 seconds, may be another parameter.

The parameters may each have criteria (such as a minimum magnitude, a maximum magnitude, or a range) for acceptable shaking that is chosen based on the particular parameters of the pharmaceutical components, such as viscosity and volume of each component, whether solid particles are present, the viscosity and miscibility of the components, and like parameters.

Examples of criteria against which the above shaking parameters may be assessed are provided below for illustration. For mixing pharmaceutical components, such as those in a paliperidone palmitate composition, the criteria relating to force or acceleration may be in one of the following ranges along the L-L axis: 2 g to 15 g, 5 g to 12 g, and 7 g to 10 g or at a threshold of 3 g, 4 g, 5 g, 6 g, 7 g, 8 g, 9 g or 9.3 g. Optionally, the force or acceleration criteria may be chosen as a minimum value. And the criteria relating to longitudinal shaking may be substantially along the longitudinal axis, or within the range +/−2 degrees relative to longitudinal axis L-L, +/− degrees relative to longitudinal axis L-L, or +/−30 degrees relative to longitudinal axis L-L. The criteria relating to verticality of housing 120 over the shaking motion may be substantially vertical, or within the range +/−2 degrees relative to vertical, +/−degrees relative vertical, or +/−30 degrees relative to vertical. The rate of shaking may be between 2 and 6 cycles per second, or between 3 and 5 cycles per second, or approximately 4 cycles per second. The particular criteria for parameters for other components or other pharmaceutical compounds may be chosen according to particular attributes of the components to be mixed, as will be understood by persons familiar with the mixing requirements of pharmaceutical components.

The controller 150/350/450 may sample data from motion data and orientation data from the motion sensor(s), such as the corresponding accelerometer and magnetometer at a predetermined rate, such as every 10 milliseconds. Alternatively, in embodiments having only an accelerometer, controller 150/350/450 may sample motion data and orientation date from only the accelerometer (that is, with no data points from a magnetometer). For each data point sampled, the parameter associated with the sampled data may be give a satisfactory or “good” condition or an unsatisfactory or “bad” condition. In this regard, if an individual motion data point and/or individual orientation data point is within a predetermined range, then that data point is stored as satisfactory. If the individual data point is outside of the predetermined range, then that data point is stored as unsatisfactory. The threshold of the shaking criteria for the chosen parameters may be met by a minimum number of data points that meet the satisfactory condition over a predetermined time period or other measure, as explained more fully below in the description of operation of system 10.

A number of factors may affect the efficacy or sufficiency of the shaking in achieving mixing of pharmaceutical components. For example, a user who shakes the device 110 vigorously or “harder” may be causing a greater acceleration of the device 110 at either end of the swinging motion. This greater acceleration and harder shaking may cause faster mixing of the components as the forces which they experience will be greater, causing better dispersion. Although “harder” shaking may lead to better mixing, it is also possible that for a particular composition of pharmaceuticals the added benefit of “harder” shaking above a certain threshold may be diminishing or even counterproductive. Hence, the device 110 may only require a certain threshold to be met and then record the shaking activity from that threshold.

Orientation of the elements 120/320/410 may affect the mixing of the pharmaceutical components. For example, the user may be shaking the device 110/310/410 with an inordinate magnitude (that is, outside of the predetermined limit) of side-to-side or off-axis movement, such as in the plane of A1-A1 A2-A2, instead of along longitudinal axis L-L. Output from the corresponding accelerometer and/or magnetometer may detect this side-to-side or off-axis shaking motion. As explained more fully herein, some side-to-side or off-axis movement may be acceptable, which can be reflected in the threshold limit.

Orientation of housing 120/320/410 generally with the “tip-up” may enhance mixing of the pharmaceutical components. For example, in circumstances in which pharmaceutical components includes particles, shaking an administration device (such as a syringe) in a “tip-down” orientation may compact particles around the entrance to the needle, thereby interfering with the injection process and/or making injection more difficult. Shaking in a “tip-up” orientation may tend to better disperse particles, rather than moving the particles toward an entrance to the needle, thereby promoting a clear fluid path through the needle and making an injection easier. Accordingly, device 110/310/410 in its representation of or mimicking of the administration device, includes a parameter for orienting housing 120/320/410 in the “tip-up” position.

For the embodiment shown in FIGS. 1-10, it has been found in some circumstances that employing magnetometer 160 in a spaced apart relationship with vibration motor 174 is beneficial for microcontroller 150 to accurately and reliably sense orientation of housing 120 during shaking in some circumstances. The magnetometer's role in more accurately assessing shaking performance is enhanced when the magnetometer is free from electromagnetic interference from vibration motor assembly 170. The inventors have found that without orientation information from the magnetometer, the algorithm may require additional steps to address unusual circumstances in which a user grips a prior art device at an angle that can simulate gravity at some times during shaking.

Further, for embodiments of training devices (not shown in the figures) having only an accelerometer (that is, without a magnetometer), accelerometer output data may be difficult to reliably interpret in some circumstances, and thus may interfere with classifying the shake parameters (such as orientation) as good or insufficient, thereby inhibiting confidence in the coaching tip to be provided. In the embodiment shown in the FIGS. 1-10, magnetometer 160 may provide orientation data that is paired with vigor (that is, g force magnitude) data and direction data from accelerometer 156 to unambiguously classify the shake parameters, and thereby provide feedback and coaching tips to the user in real time with high confidence. Embodiments without a magnetometer and/or without a vibration motor are contemplated.

Display unit 210 includes a display 212 and communication capabilities, and may include computing and memory storage capabilities. For example, display unit 210 may be a tablet computer (such as an iPad, Windows tablet computer, or Android tablet computer), a smartphone (such as an iPhone or Android phone), and/or like portable device having communications capabilities suitable for communicating with injection training device 110 as described herein. The display unit 210 may also be a general purpose computer (such as a Windows PC, Apple PC, or the like, such as a laptop computer), or may be a device specially designed and manufactured for use with the injection training device 110, clip assembly 310, and/or apparatus 410. The display unit 210 may be configured to have software/application instructions for performing the functions described herein, including communicating with the device 110/310/410, assessing and classifying the information from the accelerometer 156 and (optionally) magnetometer 160 (such as accelerometer 156 and magnetometer 160 in the embodiment of FIGS. 1-10), providing visual indications on a display screen 212 of display unit 210, and providing haptic feedback to the user via signals to the vibration motor assembly (where present) of the device 110/310/410, thereby providing feedback to a user of device 110/310/410 relative to the efficacy of shaking of device 110/310/410, as described herein. Some or all of the software/application may also be contained in the microcontroller 150/350/450, and it is understood that the functions described herein may be performed by software, firmware, hardware, and/or a combination thereof.

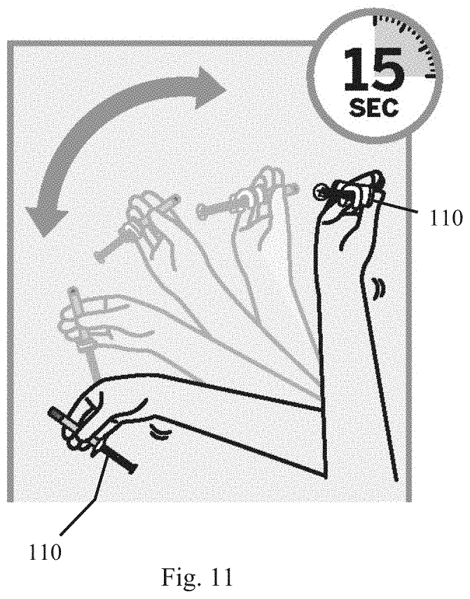

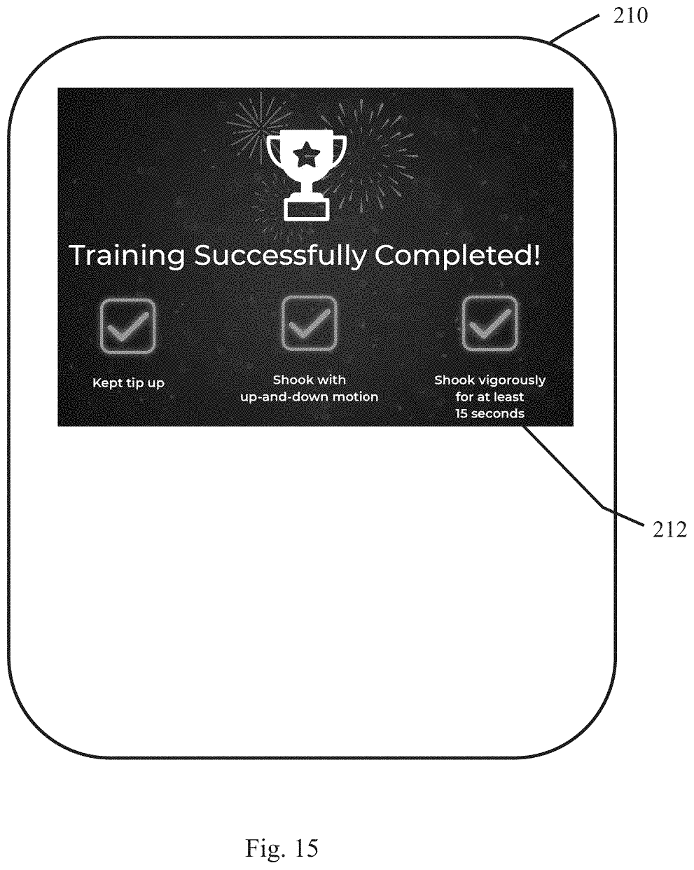

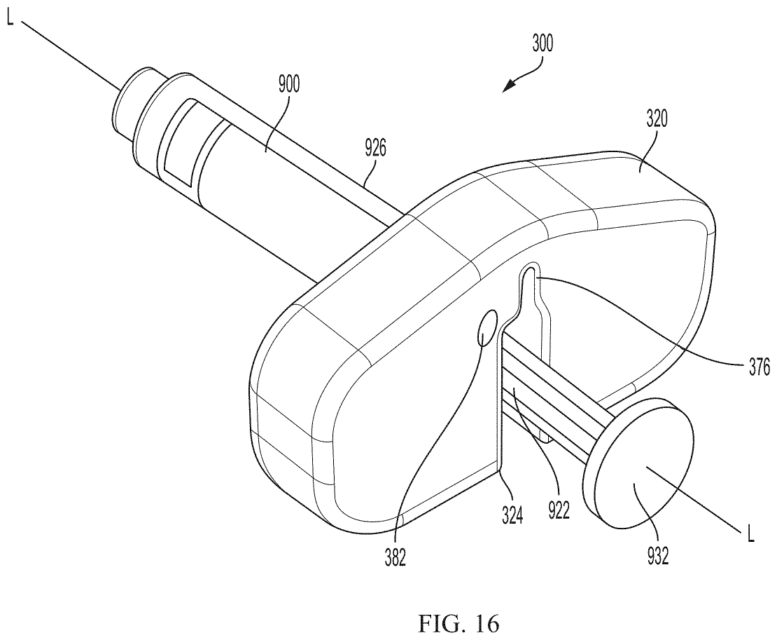

Referring to FIGS. 11 through 15 to illustrate the operation of training system 10/310/410, a user may begin by turning on display unit 210. Device 110/310/410 may be activated via software on display unit 210 such that display unit 210 sends a signal to device 110/310/410 via the communications protocol described above. Device 110/310/410 may be activated or turned on by other means, such as an on/off switch of housing 120/320/420 (not shown) or by shaking device 110/310/410 while display 210 is on.

System 10/300/400 may initially check that the user is holding housing 120 in a vertical orientations such that longitudinal axis L-L of housing 120 is within predetermined range of angles relative to vertical, and that housing 120 is in a “tip-up” orientation, by (or primarily by) assessing information from accelerometer 156 or magnetometer 160. Upon confirming a correct starting orientation in this regard, display 210 may provide a visual prompt to the user to begin shaking device 110, such as lighting LEDs 182 and/or displaying visual and/or text prompts or instructions on display 212.

The user may be prompted to begin vigorously shaking device for at least 15 seconds, as illustrated in FIG. 11. During shaking by the user, microcontroller 150 may sample data from accelerometer 156 and (optionally) magnetometer 160 at predetermined periods (such as every 10 milliseconds), according to conventional data sampling processes. Each data point may be classified as satisfactory if it satisfies the criteria corresponding to the sampled data point. For example, if a data point for g-force is within the chosen range, described above, then the data point is classified as satisfactory. Thus, a serious of data points, each one classified as satisfactory or unsatisfactory, for a given parameter are provided to controller 150.

If a quantity of satisfactory data points (that is, data points meeting the relevant criteria) within a predetermined time period is above a predetermined threshold, then the parameter corresponding to the data point is considered to have been met. Then display 212 may provide a positive visual indication, such as green up-and-down curved arrows and the word “Good,” or like positive feedback, pertaining to the parameter. If the sampled data includes an insufficient number of data points classified as satisfactory over the time period, then display 210 may show guidance or coaching tips to improve the user's shaking regarding the parameter.

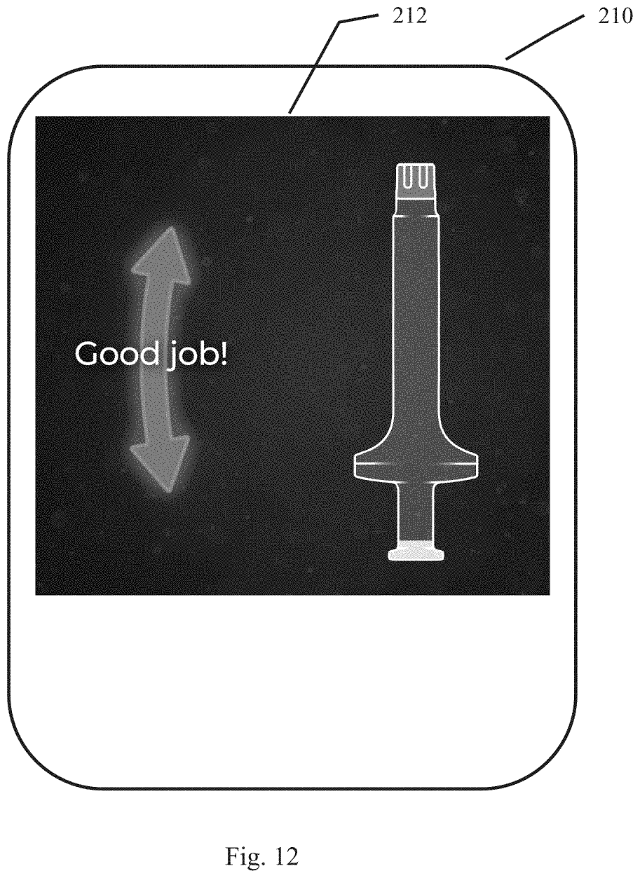

The shaking may continue for the maximum shaking window (60 seconds in the above example) while the user follows the guidance from display 212 to shake device 110 until the thresholds of all desired parameters, measured by number of good data points over a predetermined time, are met. Thus, display 212 may confirm sufficiency of the shaking and/or may provide guidance to improve the shaking sufficiency relative to the shaking parameters during the shaking (that is, in real time). If all shaking criteria meet the predetermined thresholds, display 212 may inform the user when the shaking parameters have been met over a sufficient time period to reflect successful mixing when applied to an injection device. In this regard, display 212 may provide positive visual feedback in real time to the user, such as displaying the green up-and-down curved arrow and the text “Good job!” as illustrated in FIG. 12.

Motion and orientation thresholds may be assessed by various methods, such as quantity of satisfactory data points on a running basis (such as, satisfactory data points over the prior 10 or 20 data points, over the prior 100 or 200, or 500 milliseconds, or like metric), quantity of data points over predetermined time periods (such as satisfactory data points taken in blocks over a predetermined time periods, such as every 100 or 200, or 500 milliseconds beginning with the shaking or other starting point), quantity of satisfactory data points in a row, or like methods.

For a specific example, during shaking of device 110/320/420, the microcontroller 150/350/450 may receive data from the accelerometer and (optionally) magnetometer regarding the orientation of housing 120/320/420 during shaking, such as a one data point every 10 milliseconds, which data point is classified as either a good orientation data point or an insufficient orientation data point (that is, respectively, within or outside of the desired range of angles relative to vertical). In this regard, orientation data may reflect sufficiency of the vertical orientation and a confirmation that the housing 120/320/420 is sufficiently pointed upwardly. The microcontroller 150/350/450 may also receive data from the accelerometer regarding the magnitude of force applied to housing 120/350/450 and/or the time per shaking cycle, reflected in shaking frequency in units of shaking cycles per unit time, during shaking. Each data point may be interpreted by the software or application as either a sufficient acceleration data point or an insufficient acceleration data point (that is, respectively, within or outside of the desired range of g-forces during the shaking cycle, such as at the top and/or bottom of the shaking amplitude and/or therebetween) and/or either sufficient shaking frequency or insufficient shaking frequency data point (that is, respectively, within or outside of the desired range of shaking frequency, as explained more fully below).

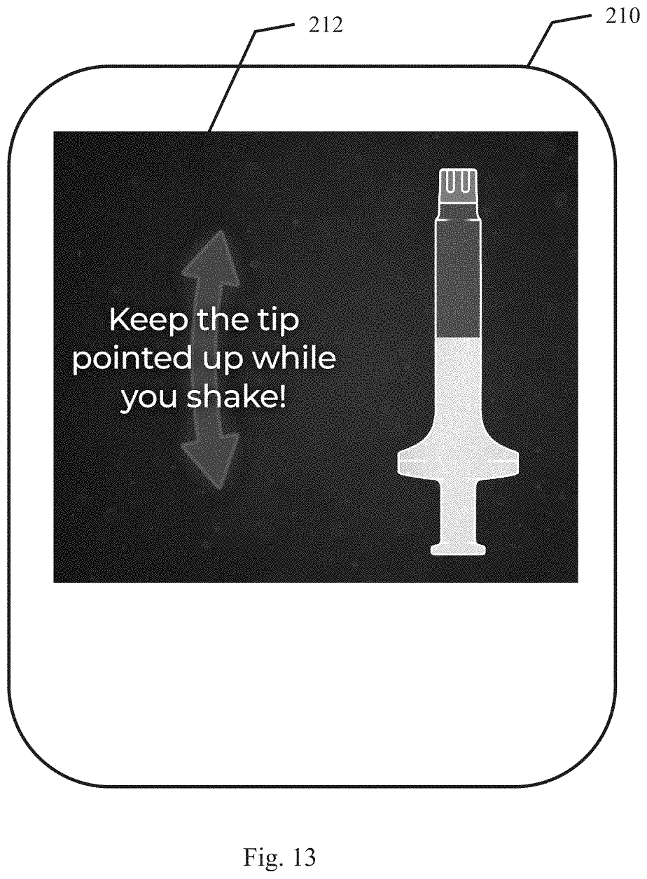

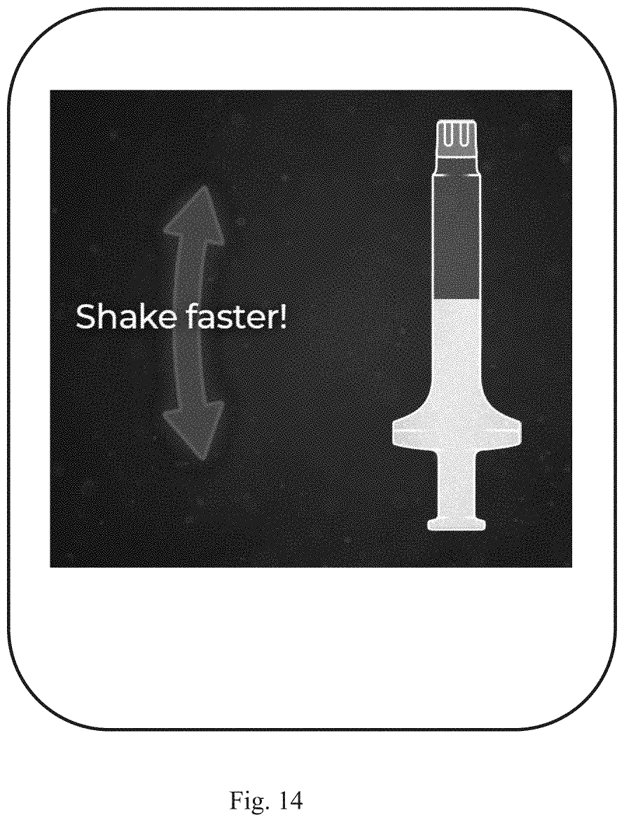

If over the short time period, there are an insufficient quantity of good data points, then display 212 may provide guidance or coaching tips for improving shaking. FIG. 13 illustrates guidance on display 212 for improving shaking in response to housing 120 being insufficiently pointed upwards, stating “Keep the tip pointed up while you shake!” with an image of the injection training device 110/320/420 in a “tip-up” orientation. If over the short time period, the data from accelerometer 156 indicates insufficient force and/or insufficient shake cycles per second, then a red up-and-down curved arrow may be shown with the text “Shake Faster!” may be displayed, as shown in FIG. 14. If over the short time period, the data from magnetometer 160 indicates insufficient vertical orientation, then the red up-and-down curved arrow may be shown with the text “Try to keep shake motion up and down!” may be shown on display 212.

Further, microcontroller and/or software on display 210 may engage the vibration motor assembly during the shaking. Controlling motor (such as motor 174), such as switching the motor on and off, may be set at a predetermined target frequency of shaking to aid the user in timing. Further, the speed of haptic feedback provided by the vibration motor assembly 170 may also be adjusted up or down to provide a haptic guidance to the user regarding the need to speed up or slow down the shaking frequency.

Upon satisfying all the required parameters relating to shaking, display 212 may indicate to the user that the shaking has been successful. In response to successful shaking on all parameters, display 212 may show green check boxes for “Kept tip up,” “Shook with up-and-down motion,” and “Shook vigorously for at least 15 seconds” under the heading “Training Successfully Completed!” as illustrated in FIG. 14. The success screen of FIG. 14 may be displayed at the end of the maximum shaking window (such as 60 seconds) or may be displayed upon satisfaction of all shaking parameters over a prior period, such as 15 seconds.

If one or more of the parameters are not met during shaking, the final screen may include the heading “Training Not Successfully Completed,” with the check boxes of FIG. 14 providing coaching tips. For example, if the parameter relating to the “tip up” is not met according to the criteria provided herein, then the left check box may show a red X, stating “Next time, keep tip up.” If the parameter relating to the orientation is not met, then the center check box may show a red X, stating “Next time, shake with up-and-down motion.” If the parameter relating to shaking vigor is not met, the left check box may show a red X, stating “Next time, vigorously shake for at least 15 seconds.”

Thus, training system 10, clip system 300, and/or apparatus 410 may be employed to train users in the mixing or pharmaceutical components. It is understood that the devices, such as clip system 300 and/or apparatus 410 may also be employed to confirm that the pharmaceutical components in the housing 320 and/or housing 420 have been sufficiently shaken. In this regard, the clip system 300 and/or apparatus 410 may provide feedback to a user prior to commencing an injection that the pharmaceutical components contained in the pharmaceutical administration device (e.g., syringe) have been adequately mixed prior to commencing an injection.

As used herein, the terms “about”, “approximately”, or “substantially” for any numerical values or ranges indicate a suitable dimensional tolerance that allows the part or collection of components to function for its intended purpose as described herein. More specifically, “about”, “approximately”, or “substantially” may refer to the range of values ±10% of the recited value, e.g. “about 90%” may refer to the range of values from 81% to 99%. In addition, as used herein, the terms “patient,” “host,” “user,” and “subject” refer to any human or animal subject and are not intended to limit the systems or methods to human, animal or medical use, although use of the subject invention in a human patient represents a preferred embodiment. Reference to “g” in terms of acceleration means 9.81 ms⁻², approximately 10 ms⁻². The phrases “in the housing” and “within the housing,” and like phrases, are intended to be broadly interpreted to encompass structures that are wholly encased inside the housing, structures that are partly encased inside the housing and/or visible, and structures that are at least partly outside of the housing and attached to the housing. The phrase “such as” is used to provide examples without limitation. The term “software” refers to computerized functions and/or instructions, and it is understood that firmware, hardware, and/or a combination of software, firmware, and hardware may be employed for the functions and/or instructions.

The present invention has been described using the structure and function of the above embodiments of an injection training device and a display unit. The present invention is not intended to be limited to the structure and/or function described herein unless set out in the claims. Moreover, the terms for the components employed to illustrate the embodiments described herein are meant to be broadly interpreted to encompass all types. The detailed disclosure should be read with reference to the drawings, in which like elements in different drawings are identically numbered. The drawings depict particular embodiments and are not intended to limit the scope of the invention. This detailed disclosure illustrates by way of example, not by way of limitation, the principles of the invention. This disclosure clearly enables one skilled in the art to make and use the invention, and describes several embodiments, adaptations, variations, alternatives and uses of the invention, including what is presently believed to be the best mode of carrying out the invention.

We claim:

1. A system for training a user in proper mixing of pharmaceutical components in a syringe, the system comprising:
an injection training device including:
a housing;
a controller disposed in the housing;
one or more motion sensors in communication with the controller, wherein the one or more motion sensors is configured to detect and output, to the controller, (i) motion information regarding motion of the housing and (ii) orientation information regarding orientation of the housing; and
a display unit that is spaced apart from the injection training device, the display unit being in communication with the controller;
wherein, when the housing is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

2. The system of claim 1, wherein the injection training device further comprises a vibration motor disposed in the housing, the vibration motor being adapted for providing haptic feedback to the user.

3. The system of claim 2, wherein the haptic feedback is a vibration motor output at a predetermined frequency that relates to a desired shaking frequency.

4. The system of claim 2, wherein the one or more motion sensors is a multi-axis accelerometer adapted to provide the motion information and the orientation information.

5. The system of claim 4, wherein the one or more motion sensors includes an accelerometer adapted for providing the motion information and a magnetometer adapted for providing the orientation information.

6. The system of claim 5, wherein the controller is a microcontroller, and the microcontroller, the accelerometer, and the magnetometer are mounted on a printed circuit board, and wherein the vibration motor is spaced apart from the magnetometer, thereby diminishing magnetic interference between the vibration motor and the magnetometer.

7. The system of claim 6, wherein the display unit is one of a tablet, a smartphone, and a computer.

8. The system of claim 7, wherein the microcontroller is adapted for sampling motion output data points from the accelerometer and orientation output data points from the magnetometer multiple times per second, wherein if a predetermined quantity of motion output data points over a predetermined time period and a predetermined quantity of orientation output data points over the predetermined time period satisfy predetermined thresholds, the shaking is determined to be satisfactory.

9. The system of claim 8, wherein the housing forms a barrel, a finger flange body, and a plunger body; the barrel defining a longitudinal axis of the injection training device, the printed circuit board being disposed in the finger flange body, the plunger body extending from the finger flange body along the longitudinal axis, and at least a portion of the vibration motor being disposed in the plunger body.

10. The system of claim 9, further comprising a battery assembly that is at least partly disposed in the barrel and that is adapted for powering the microcontroller.

11. The system of claim 10, wherein the display unit is adapted to visually indicate to the user a positive indication for the at least one characteristic of the shaking upon meeting the predetermined threshold in real time, and wherein the display unit is adapted to visually indicate to the user a negative indication for the at least one characteristic of the shaking upon failing to meet the predetermined threshold in real time.

12. The system of claim 11, wherein the at least one characteristic of the shaking includes motion, frequency, and/or vertical orientation of the shaking.

13. The system of claim 12, wherein the injection training device further includes at least one light adapted for indicating shaking efficacy by the user.

14. The system of claim 1, wherein the injection training device includes a clip assembly and a drug injection device, and wherein the clip assembly defines the housing.

15. The system of claim 14, wherein the one or more motion sensors is a multi-axis accelerometer adapted to provide the motion information and the orientation information.

16. The system of claim 14, wherein the one or more motion sensors includes an accelerometer adapted for providing the motion information and a magnetometer adapted for providing the orientation information.

17. The system of claim 14, wherein the display unit is one of a tablet, a smartphone, and a computer.

18. The system of claim 17, wherein the controller is a microcontroller adapted for sampling motion output data points and orientation output data points multiple times per second, wherein if a predetermined quantity of motion output data points over a predetermined time period and a predetermined quantity of orientation output data points over the predetermined time period satisfy predetermined thresholds, the shaking is determined to be satisfactory.

19. The system of claim 17, wherein the display unit is adapted to visually indicate to the user a positive indication for the at least one characteristic of the shaking upon meeting the predetermined threshold in real time, and wherein the display unit is adapted to visually indicate to the user a negative indication for the at least one characteristic of the shaking upon failing to meet the predetermined threshold in real time.

20. A system for training a user in proper mixing of pharmaceutical components in a syringe, the system comprising:
an injection training device including:
a flexible PCB affixed to a drug injection device;
a controller disposed on the PCB;
one or more motion sensors disposed on the PCB in communication with the controller, wherein the one or more motion sensors is configured to detect and output, to the controller, (i) motion information regarding motion of the syringe and (ii) orientation information regarding orientation of the syringe; and
a display unit that is spaced apart from the injection training device, the display unit being in communication with the controller;
wherein, when the syringe is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

21. The system of claim 20, wherein the one or more motion sensors is a multi-axis accelerometer adapted to provide the motion information and the orientation information.

22. The system of claim 21, wherein the one or more motion sensors includes an accelerometer adapted for providing the motion information and a magnetometer adapted for providing the orientation information.

23. The system of claim 22, wherein the display unit is one of a tablet, a smartphone, and a computer.

24. The system of claim 23, wherein the controller is a microcontroller adapted for sampling motion output data points and orientation output data points multiple times per second, wherein if a predetermined quantity of motion output data points over a predetermined time period and a predetermined quantity of orientation output data points over the predetermined time period satisfy predetermined thresholds, the shaking is determined to be satisfactory.

25. The system of claim 24, wherein the display unit is adapted to visually indicate to the user a positive indication for the at least one characteristic of the shaking upon meeting the predetermined threshold in real time, and wherein the display unit is adapted to visually indicate to the user a negative indication for the at least one characteristic of the shaking upon failing to meet the predetermined threshold in real time.

26. A system for training a user in proper mixing of pharmaceutical components in a syringe, the system comprising:
a clip assembly including:
a housing adapted to be removably affixed to a drug injection device;
a controller disposed in the housing;
one or more motion sensors in communication with the controller, wherein the one or more motion sensors is configured to detect and output, to the controller, (i) motion information regarding motion of the housing and (ii) orientation information regarding orientation of the housing; and
a display unit that is spaced apart from the injection training device, the display unit being in communication with the controller;
wherein, when the housing is subjected to shaking, the controller receives the motion information and the orientation information, and wherein, based on the received information, the display unit provides a visual indication of whether at least one characteristic of the shaking meets a predetermined threshold, and the display unit provide coaching tips to the user if the at least one characteristic of the shaking fails to meet the predetermined threshold.

27. A method for training a user in proper mixing of pharmaceutical components in a syringe, the method comprising the steps of:
shaking an injection training device that includes a controller and one or more sensors;
receiving, into the controller, motion data points from the one or more sensors in response to the shaking step;
receiving, into the controller, orientation data points from the one or more sensors in response to the step of shaking the injection training device;
classifying each one of the motion data points as satisfactory or unsatisfactory based on whether the motion data point meets a predetermined motion threshold;
classifying each one of the orientation data points as satisfactory or unsatisfactory based on whether the orientation data point meets a predetermined orientation threshold,
determining whether at least one characteristic of the shaking is satisfactory based on (i) a predetermined quantity of satisfactory motion data points meeting a predetermined threshold and/or (ii) a predetermined quantity of satisfactory orientation data points meeting a predetermined threshold; and
based on the step of determining whether at least one characteristic of the shaking is satisfactory, displaying, on a display unit that is spaced apart from the injection training device, a visual indication of whether the at least one characteristic is satisfactory, and providing coaching tips to the user in real time.

28. The method of claim 27, wherein the step of receiving motion data points includes sampling motion data points from the motion sensor at a rate of multiple times per second, and the step of receiving orientation data points includes sampling orientation data points from the orientation sensor at a rate of multiple times per second.

29. The method of claim 28, further comprising a step of classifying each one of the motion data points as sufficient or insufficient based on predetermined parameters, and the step of classifying each one of the orientation information data points as sufficient or insufficient based on predetermined parameters.

30. The method of claim 29, further comprising a step of assessing whether a quantity of the motion information data points and a quantity of orientation information data points within a predetermined time period are sufficient to satisfy predetermined criteria.

31. The method of claim 30, further comprising a step of providing haptic feedback relating to a desired shaking frequency to a user via a vibration motor that is spaced apart from the magnetometer.

32. The method of claim 31, wherein the injection training device includes a housing having a barrel, a finger flange body, and a plunger body that at least partly houses the vibration motor, the housing defining a longitudinal axis for defining an up-and-down shaking axis.

33. The method of claim 32, wherein the step of displaying includes providing coaching tips relating to the at least one characteristic of the shaking in real time.

34. The method of claim 33, wherein the at least one characteristic of the shaking includes motion, frequency, and/or vertical orientation of the shaking.

35. The method of claim 34, further comprising a step of activating lights on the housing based on at least one characteristic of the shaking.

36. The method of claim 27, wherein the step of shaking the injection training device includes shaking a clip assembly affixed to a drug injection device, wherein the clip assembly defines the housing.

37. The method of claim 36, wherein the steps of receiving the motion data points and receiving the orientation data points include receiving the motion data points and receiving the orientation data points from a multi-axis accelerometer.

38. The method of claim 37, wherein the step of receiving the motion data points includes receiving the motion data points from an accelerometer and the step of receiving the orientation data points includes receiving the orientation data points from a magnetometer.

39. The method of claim 38, wherein the steps of displaying the visual indication and providing the coaching tips includes displaying the visual indication and providing the coaching tips on one of a tablet, a smartphone, and a computer.

40. The method of claim 39, wherein the steps of receiving the motion data points and receiving the orientation data points includes receiving the motion data points and receiving the orientation data points in a microcontroller multiple times per second, and, if a predetermined quantity of motion output data points over a predetermined time period and a predetermined quantity of orientation output data points over the predetermined time period satisfy predetermined thresholds, displaying satisfaction of the shaking parameters on the display unit.

41. The method of claim 36, further comprising a step of removing the clip assembly from the syringe and coupling the clip assembly to another syringe to enable the shaking step to be performed on the other syringe.

42. The method of claim 33, wherein the step of shaking the injection training device includes shaking a syringe having a smart label affixed thereto.

43. The method of claim 42, wherein the steps of receiving the motion data points and receiving the orientation data points include receiving the motion data points and receiving the orientation data points from a multi-axis accelerometer.

44. The method of claim 43, wherein the step of receiving the motion data points includes receiving the motion data points from an accelerometer and the step of receiving the orientation data points includes receiving the orientation data points from a magnetometer.

45. The method of claim 44, wherein the steps of displaying the visual indication and providing the coaching tips includes displaying the visual indication and providing the coaching tips on one of a tablet, a smartphone, and a computer.

46. The method of claim 45, wherein the steps of receiving the motion data points and receiving the orientation data points includes receiving the motion data points and receiving the orientation data points in a microcontroller multiple times per second, and, if a predetermined quantity of motion output data points over a predetermined time period and a predetermined quantity of orientation output data points over the predetermined time period satisfy predetermined thresholds, displaying satisfaction of the shaking parameters on the display unit.
